SEQUENCE LISTING (5 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 224 aa, Homo sapiens
ESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNE

SEQ ID NO: 3 - DNA, 1102 bp, Homo sapiens
gcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatactaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 4 - protein, 310 aa, Homo sapiens
CKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVT

SEQ ID NO: 5 - protein, 298 aa, Homo sapiens
ESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKA

(19) United States
(12) Patent Application Publication
(10) Pub. No.: US 2005/0063944 A1
(43) Pub. Date: Mar. 24, 2005

(54) Method of inducing maturation of dendritic cells and uses therefor

(76) Inventors: Jian Li, Secane, PA (US); Lamine Mbow, Malvern, PA (US); Theresa J. Goletz, King of Prussia, PA (US); David Peritt, Cynwyd, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/666,490

(22) Filed: Sep. 19, 2003

(51) Int. Cl. A61K 38/19; A61K 38/20

(52) U.S. Cl. 424/85.1; 424/85.2

(57) ABSTRACT

The invention relates to the induction of responses relating to the maturation of dendritic cells, using IL-18 and IL-18 muteins, and compounds, compositions, methods of making and using thereof, including therapeutic methods and products.

### PRIORITY APPLICATION

This application claims priority to and entirely incorporates by reference U.S. provisional patent application No. 60/412,145, filed Sep. 19, 2002.

### FIELD OF THE INVENTION

The invention, in the field of biotechnology, relates to the induction of responses relating to the maturation of dendritic cells, using IL-18 and IL-18 muteins, and compounds, compositions, methods of making and using thereof, including therapeutic methods and products.

### BACKGROUND OF THE INVENTION

Interleukin (IL)-18 is an IL-1-like proinflammatory cytokine that is thought to have various effects on T-cell activation. IL-18 is also thought to play a role in the T-cell-helper type I (Th1) response, primarily by its apparent ability to induce IFN-γ production in T cells and natural killer (NK) cells. IL-18 is also thought to play a role in the induction of gene expression and synthesis of tumor necrosis factor (TNFα), IL-1β, Fas ligand, GM-CSF, and several chemokines, depending on the cell type responding and the conditions used.

IL-18 was initially designated “interferon-γ inducing factor (IGIF)” immediately after the identification of its activity and cloning from mice. This designation was changed later into IL-18 and the cloning of the cDNA described. Mature IL-18 consists of 157 amino acids. In vivo, IL-18 is thought to be formed by cleavage of an 193 amino acid precursor by the ILAP converting enzyme (IL-1beta-converting enzyme, ICE, caspase-1.

IL-18 receptors comprise at least two subunits: IL-18R (also known as IL-1R-related protein, IL-1Rrp, IL-18Rα, 2FI or the “binding chain”) and AcPL (also known as accessory protein-like, L-18-AcPL, IL-18Rβ or the “signaling chain”), also a member of the IL-1R family. The receptor complex recruits the IL-1R-activating kinase (IRAK) and TNFR-associated factor-6 (TRAF-6) which phosphorylates nuclear factor kB (NFkB)-inducing kinase (NIK) with subsequent activation of NFkB.

IL-18 is produced by various cell types such as macrophages, peritoneal exudate cells, and microglial cells among others. Peripheral blood mononuclear cells (PBMC) can secrete pro-IL-18. Dendritic cells express IL-18 mRNA and produce mature IL-18.

IL-18 has multiple biological activities such as sustaining development of Th1 phenotype, synergy with IL-12 in the production of IFN gamma and enhancement of CC and CXC chemokine production by T and NK cells. IL-18 was initially thought of primarily as a co-stimulant for Th1 cell production of IFN-gamma, IL-2 and GM-CSF, and as a co-stimulant for FAS ligand-mediated cytotoxicity of murine natural killer cell whereas this effect was not seen in Th2 cells. More recently, the role of IL-18 in Th2 cell stimulation has been noted despite the lack of IL-18R on these cells. IL-18 was shown to be capable of inducing Th2 related cytokines from T, NK and basophils/mast cells.

IL-18 shares biological similarity with IL-12, also a strong cofactor for Th1 T-cell development. IL-18 enhances T cell proliferation, apparently through an IL-2-dependent pathway, enhances Th1 cytokine production in vitro and exhibits synergism when combined with IL-12 in terms of enhanced IFN-gamma production and NK cell activity.

A recent study demonstrated that IL-18 was also a strong co-factor for the expression of a Th2 cytokines such as IL-4, IL-5, and IL-13 and that IL-18 and T-cell receptor-mediated stimulation could induce naïve CD4+ T-cells to develop into IL-4 producing cells in vitro.

The ability of IL-18 but not IL-12 to induce IFN-gamma in the human myelomonocytic cell line, KG-1 cell was reported and later that IL-18 induces IFN-gamma production and ICAM-1 expression on KG-1 cells by signaling through NFkappaB. IL-18 induces IFNgamma, ICAM-1 and CD95 expression on primary murine macrophages as well as IL-18 production itself. These findings indicate that monocytes express and signal via IL-18R. On the other hand, IL-18 was shown to induce Fas-ligand expression on the human myelomonocytic cell line KG-1 and induce apoptosis of Fas-expressing KG-1 cells.

Dendritic cells (DC) originate from hemopoietic stem cells and subsequently migrate to and reside in both lymphoid and non-lymphoid tissues, where they are able to capture and process antigen. Immature DC (iDC) have low ability to stimulate naïve T cells despite their high antigen processing capacity. Mature DC express high levels of MHC II and accessory cell molecules and become potent stimulators of naïve T cells. Immature DC mature and migrate from nonlymphoid tissues to lymph follicles to become follicular dendritic cells (FDC) after encounter of specific stimuli, such as by inflammatory mediators. Thus, migration and maturation of DC is one of the critical steps in the immune response.

IL-4, a Th2 cytokine, acts synergistically with IL-12 on IFN-gamma production in mature dendritic cells during T cell-DC interaction upon antigen presentation. Furthermore, that IL-18 appears to enhance IFN-gamma production in mature dendritic cells through an intracellular pathway distinct from that of IL-4. However, stimulators of the maturation process from hematopoietic cells are less well understood.

The mechanism of action for dendritic cell antigen presentation has also been explored. Antigen uptake by dendritic cells via Fcg receptors results in functional augmentation of antigen presentation and T cell proliferation in an in vitro sheep system. Fcg receptors induce dendritic cell maturation and promote efficient MHC class I-restricted presentation of peptides from exogenous, immunoglobulin (Ig) complexed antigens in the mouse system.

Thus, there remains a need to discover methods for utilizing dendritic cells to treat human diseases. The promise of dendritic cell-based approaches to treat diseases; such as, but not limited to, cancer, GvHD, infectious diseases caused by pathogens, and others, and to modulate allergic responses, underscores the need to develop autologous cell based approaches as effective therapeutic treatments.

### SUMMARY OF THE INVENTION

It is the object of this invention to provide a method of modulating (e.g., enhancing or suppressing) at least one immune response in subjects in need thereof.

The invention relates to the use of exogenously supplied IL-18, an active analog of IL-18, or an IL-18R agonist to cause maturation of dendritic cells from myeloid precursors. The invention further relates to the use of IL-18, an active analog of IL-18, or IL-18R agonist to activate DC for use in a mammal to stimulate the immune response in said mammal.

In a particular preferred embodiment, DC activated extracorporally (ex vivo) by exogenously supplied human rIL-18 or an analog, are administered to a patient requiring such treatment.

The activated DC may be used to augment and direct the immune response of the host to a tumor or pathogens. The activated DC may be used to modulated the host allergic response as in asthma. The IL-18-activated DC may be used to enhance maturation of PBSC in patients previously treated with therapies that cause a reduction in said PBSC, including patients undergoing high-dose chemotherapy.

In a second aspect, the invention provides a method for treating a patient suffering from a disease associated with an antigen, comprising administering to the patient a composition a matured autologous dendritic cell loaded with the antigen, and a dendritic cell autologous to the patient, wherein the patient administered the composition receives a therapeutic benefit. Preferably, the patient is a human.

In a third aspect, the invention provides a process of preparing matured DC using exogenously supplied IL-18 or combination of maturation inducing agents including IL-18. The process comprises methods of selecting specific subpopulations of DC derived from monocytes or bone marrow precursors.

In a fourth aspect, the invention provides a therapeutic composition comprising a substantially purified IL-18 matured dendritic cell that is specific for an antigen associated with a disease. In preferred embodiments, IL-18 matured dendritic cell is administered with the antigen. In certain embodiments of the fourth aspect of the invention, administration of the composition to a patient suffering from the disease provides the patient a therapeutic benefit. In certain embodiments, administration of the composition to a patient suffering from the disease provides the patient a therapeutic benefit, wherein the dendritic cell is autologous to the patient. Preferably, the patient is a human.

### DETAILED DESCRIPTION OF THE INVENTION

Dendritic cells (DCs) are professional antigen presenting cells that are critical for the initiation of T cell-dependent immune response by presenting peptide in the context of MHC along with appropriate costimulation. Immature DCs exhibit a higher capacity for antigen uptake and processing than mature DCs, but they unable to prime naïve T cells. Mature DCs increase their expression of CD83, CD40, CD80, CD86, ICAM-1, CCR7 and acquire the capacity to activate naïve, and memory lymphocytes. Heretofore, it was known that DC maturation can be triggered by a variety of factors, including LPS, TNFα, IL-1β, PGE₂ and CD40 ligand.

It is well known that IL-18 acts on T and NK cells to induce the production of IFNgamma, however, the effect of IL-18 on DCs maturation has not yet been elucidated. The studies described herein elucidate the role of IL-18 on DC maturation.

CD83 is an inducible glycoprotein expressed predominantly by mature DC, including Langerhans cells and dermal DC within skin. Expression of membrane CD83 is widely used as a marker of mature DC. CD83 shows highly restricted cellular expression and has significant homologies with the B7 ancestral gene family that includes B7-1 and B7-2. The function of human CD83 and its ligand has yet to be established. Scholler et al. suggested that CD83 mediates adhesion of DC to circulating monocytes and to a fraction of activated T cells or stressed T cells by a specific binding of CD83 to a 72-kDa ligand (Scholler, N., et al. J. Immunol. 166:3865-3872, 2001). Up-regulation of CD83, ICAM-1, LT, GRO-gamma genes as well as CD83, ICAM-1 and other co-stimulatory proteins by IL-18 indicate changes consistent with a process of DC maturation.

Up-regulated CD83 by IL-18 is related to functional maturation of DCs. The priming ability of DCs on primary T cell responses is acquired upon encounter with maturation stimuli. LPS, TNFα are been shown to lead to DC maturation. DC maturation is accompanied by high levels of CD40, CD80 and CD83 expression. As an important regulator for innate and acquired immune response, the role of IL-18 on DC maturation has been little explored. Our investigation of changes in key gene and protein expression levels has identified a role for IL-18 on DC maturation.

IL-18 Biologically Active Compounds

Any compound having suitable IL-18 biological activity for maturing DC can be used according to the present invention. A non-limiting example is the use of IL-18 proteins or receptors and muteins or variants thereof having suitable biological activity for maturing DC cells as described herein.

Nucleic Acid Molecules

Using the information provided herein, such as the nucleotide sequences encoding at least 70-100% of the contiguous amino acids of at least one of SEQ ID NOS: 1-2, specified fragments, variants or consensus sequences thereof, or a deposited vector comprising at least one of these sequences, a nucleic acid molecule of the present invention encoding at least one IL18 or IL-18R protein can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as theanti-sense strand.

Isolated nucleic acid molecules of the present invention can include nucleic acid molecules comprising an open reading frame (ORF), optionally with one or more introns; nucleic acid molecules comprising the coding sequence for an IL-18 or IL-18R protein or variable region; and nucleic acid molecules which comprise a nucleotide sequence substantially different from those described above but which, due to the degeneracy of the genetic code, still encode at least one IL18 or IL-18R protein as described herein and/or as known in the art. Of course, the genetic code is well known in the art. Thus, it would be routine for one skilled in the art to generate such degenerate nucleic acid variants that code for specific IL18 or IL-18R proteins of the present invention. See, e.g., Ausubel, et al., supra, and such nucleic acid variants are included in the present invention.

In another aspect, the invention provides isolated nucleic acid molecules encoding a(n) IL18 or IL-18R protein having an amino acid sequence as encoded by the nucleic acid contained in the plasmid deposited as designated clone names ______ and ATCC Deposit Nos. ______, respectively, deposited on ______.

As indicated herein, nucleic acid molecules of the present invention which comprise a nucleic acid encoding an IL18 or IL-18R protein can include, but are not limited to, those encoding the amino acid sequence of an IL-18 or IL-18R fragment, by itself; the coding sequence for the entire protein or a portion thereof, the coding sequence for an protein, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an protein can be fused to a marker sequence, such as a sequence encoding a peptide that facilitates purification of the fused protein comprising an protein fragment or portion.

Polynucleotides which Selectively Hybridize to a Polynucleotide as Described Herein

The present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably at least 85% or 90% full-length sequences, and more preferably at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this invention will encode at least a portion of an protein encoded by the polynucleotides described herein. The polynucleotides of this invention embrace nucleic acid sequences that can be employed for selective hybridization to a polynucleotide encoding an protein of the present invention. See, e.g., Ausubel, supra; Colligan, supra, each entirely incorporated herein by reference.

Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra)

Nucleic Acid Screening and Isolation Methods

A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that usesanti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

Synthetic Methods for Constructing Nucleic Acids

The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The present invention further provides recombinant expression cassettes comprising a nucleic acid of the present invention. A nucleic acid sequence of the present invention, for example a cDNA or a genomic sequence encoding an protein of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

Vectors And Host Cells

The present invention also relates to vectors that include isolated nucleic acid molecules of the present invention, host cells that are genetically engineered with the recombinant vectors, and the production of at least one IL18 or IL-18R protein by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one protein of the present invention can be expressed in a modified form, such as a fusion protein, and can include not only secretion signals, but also additional heterologous functional regions. For instance, a region of additional amino acids, particularly charged amino acids, can be added to the N-terminus of an protein to improve stability and persistence in the host cell, during purification, or during subsequent handling and storage. Also, peptide moieties can be added to an protein of the present invention to facilitate purification. Such regions can be removed prior to final preparation of an protein or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the present invention.

Alternatively, nucleic acids of the present invention can be expressed in a host cell by turning on (by manipulation) in a host cell that contains endogenous DNA encoding an protein of the present invention. Such methods are well known in the art, e.g., as described in U.S. Pat. Nos. 5,580,734,5,641,670, 5,733,746, and 5,733,761, entirely incorporated herein by reference.

Illustrative of cell cultures useful for the production of the proteins, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VPI intron from SV40 (Sprague, et al., J. Virol. 45:773-781 (1983)). Additionally, gene sequences to control replication in the host cell can be incorporated into the vector, as known in the art.

Purification of an Protein

A IL18 or IL-18R protein can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Proteins of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the protein of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

IL18 OR IL-18R PROTEINS

The isolated proteins of the present invention comprise at least one protein and/or protein amino acid sequence disclosed or described herein encoded by any suitable polynucleotide, or any at least one isolated or prepared protein protein. Preferably, the at least one protein has at least one IL18 or L-18R activity and the at least one protein binds human IL18 or IL-18R and, thereby partially or substantially modulates at least one structural or biological activity of at least one IL18 or IL-18R protein.

As used herein, the term “IL18 or IL-18R protein” refers to a protein as described herein that has at least one IL8 or IL-18R-dependent activity, such as 5-10000%, of the activity of a known or other IL18 or IL-18R protein or active portion thereof, preferably by at least about 10, 20, 30, 40, 50, 55, 60, 65, 70, 75, 80, 85, 90, 91, 92, 93, 94, 95, 96, 97, 98, 99, 100% or more, depending on the assay. The capacity of a IL18 or IL-18R protein to have at least one IL18 or IL-18R-dependent activity is preferably assessed by at least one suitable IL18 or IL-18R protein or receptor assay, as described herein and/or as known in the art.

Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g, charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Amino Acid Codes

The amino acids that make up IL18 or IL-18R proteins of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE LETTER | THREE LETTER | | |
|---|---|---|---|
| CODE | CODE | NAME | THREE NUCLEOTIDE CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | H | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, CGG, CGU |
| S | Ser | Serine | AGC, AGU, UCA, UCC, UCG, UGU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

An IL18 or IL-18R protein of the present invention can include one or more amino acid substitutions, deletions or additions, either from natural mutations or human manipulation, as specified herein.

Of course, the number of amino acid substitutions a skilled artisan would make depends on many factors, including those described above. Generally speaking, the number of amino acid substitutions, insertions or deletions for any given L18 or L-18R protein, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an IL18 or IL-18R protein of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one IL18 or IL-18R neutralizing activity. Sites that are critical for antibody binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

IL18 or IL-18R proteins of the present invention can include, but are not limited to, at least one portion, sequence or combination selected from 3-100 to all of the contiguous amino acids of at least one of SEQ ID NOS:1-2.

Non-limiting CDRs or portions of IL18 or IL-18R proteins of the invention that can enhance or maintain at least one of the listed activities include, but are not limited to, any of the above polypeptides, further comprising at least one mutation corresponding to at least one substitution selected from the group consisting of at least one of extracellular, intracellular, soluble, at least 10 contiguous amino acids, and the like, extracellular, intracellular, soluble, at least 10 contiguous amino acids, and the like, and/or.

Non-limiting variants that can enhance or maintain at least one of the listed activities include, but are not limited to, any of the above polypeptides, further comprising at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe21; Val22 for Ile22; Ile66 for Val66; Thr72 for Ser72; Phe148 for Ser148; Glu4 for Lys4; Ile6 for Glu6; Asp8 for Lys8; ILe 13 for Arg 13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu 133; Ala134 for Phe134; Thr150 for Met150; Ser151 for Phe151, of at least one of SEQ ID NOS:1-2.

A(n) IL18 or IL-18R protein can further optionally comprise a polypeptide of at least one of 70-100% of the contiguous amino acids of at least one of SEQ ID NOS:1-2 or any variant thereof.

In one embodiment, the amino acid sequence of a IL18 or IL-18R protein or antibody has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of SEQ ID NOS:1-2. Preferably, 70-100% amino acid identity (i.e., 90, 91, 92, 93, 94, 95, 96, 97, 98, 99, 100 or any range or value therein) is determined using a suitable computer algorithm, as known in the art.

The proteins of the present invention, or specified variants thereof, can comprise any number of contiguous amino acid residues from an antibody of the present invention, wherein that number is selected from the group of integers consisting of from 10-100% of the number of contiguous residues in an IL18 or IL-18R protein or antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active protein or antibody of the present invention. Biologically active proteins have a specific activity at least 20%, 30%, or 40%, and preferably at least 50%, 60%, or 70%, and most preferably at least 80%, 90%, or 95%-100% of that of the native (non-synthetic), endogenous or related and known protein or antibody. Methods of assaying and quantifying measures of enzymatic activity and substrate specificity, are well known to those of skill in the art.

In another aspect, the invention relates to IL18 or IL-18R proteins of the invention, as described herein, which are modified by the covalent attachment of a moiety. Such modification can produce a IL18 or IL-18R protein or anibody with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified proteins of the invention can comprise one or more organic moieties that are covalently bonded, directly or indirectly, to the protein. Each organic moiety that is bonded to the protein or antibody of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a IL18 or IL-18R protein modified by the covalent attachment of polylysine is encompassed by the invention. Hydrophilic polymers suitable for modifying proteins of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein or antibody of the invention has a molecular weight of about 800 to about 150,000 Daltons as a separate molecular entity. For example PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying proteins of the invention can be saturated or can contain one or more units of unsaturation. Fatty acids that are suitable for modifying proteins of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human proteins can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

Modified proteins of the invention can be produced by reacting the protein or antibody with a modifying agent. For example, the organic moieties can be bonded to the protein in a non-site specific manner by employing an amine-reactive modifying agent, for example, an NHS ester of PEG. Modified IL18 or IL-18R proteins can also be prepared by reducing disulfide bonds (e.g., intra-chain disulfide bonds) of the protein and antibody. The reduced protein and antibody can then be reacted with a thiol-reactive modifying agent to produce the modified antibody of the invention. Modified proteins comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

IL18 or IL-18R Protein Compositions

The present invention also provides at least one IL18 or IL-18R protein composition comprising at least one, at least two, at least three, at least four, at least five, at least six or more IL18 or IL-18R proteins or proteins thereof, as described herein and/or as known in the art that are provided in a non-naturally occurring composition, mixture or form. Such compositions comprise non-naturally occurring compositions comprising at least one or two IL18 or IL-18R protein amino acid sequences selected from the group consisting of 5-100% of the contiguous amino acids of SEQ ID NOS:1-2, or specified fragments, domains or variants thereof. Further preferred compositions comprise 40-99% of at least one of 70-100% of SEQ ID NOS:1-2, or specified fragments, domains or variants thereof. Such composition percentages are by weight, volume, concentration, molarity, or molality as liquid or dry solutions, mixtures, suspension, emulsions or colloids, as known in the art or as described herein.

IL18 or IL-18R protein compositions of the present invention can further comprise at least one of any suitable and effective amount of a composition or pharmaceutical composition comprising at least one IL18 or IL-18R protein to a cell, tissue, organ, animal or patient in need of such modulation, treatment or therapy, optionally further comprising at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Non-limiting examples of such cytokines include, but are not limted to, any of IL-1 to IL-23. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Such compositions can also include toxin molecules that are associated, bound, co-formulated or co-administered with at least one protein of the present invention. The toxin can optionally act to selectively kill the pathologic cell or tissue. The pathologic cell can be a cancer or other cell. Such toxins can be, but are not limited to, purified or recombinant toxin or toxin fragment comprising at least one functional cytotoxic domain of toxin, e.g., selected from at least one of ricin, diphtheria toxin, a venom toxin, or a bacterial toxin. The term toxin also includes both endotoxins and exotoxins produced by any naturally occurring, mutant or recombinant bacteria or viruses which may cause any pathological condition in humans and other mammals, including toxin shock, which can result in death. Such toxins may include, but are not limited to, enterotoxigenic E. coli heat-labile enterotoxin (LT), heat-stable enterotoxin (ST), Shigella cytotoxin, Aeromonas enterotoxins, toxic shock syndrome toxin-1 (TSST-1), Staphylococcal enterotoxin A (SEA), B (SEB), or C (SEC), Streptococcal enterotoxins and the like. Such bacteria include, but are not limited to, strains of a species of enterotoxigenic E. coli (ETEC), enterohemorrhagic E. coli (e.g., strains of serotype 0157:H7), Staphylococcus species (e.g., Staphylococcus aureus, Staphylococcus pyogenes), Shigella species (e.g., Shigella dysenteriae, Shigella flexneri, Shigella boydii, and Shigella sonnei), Salmonella species (e.g., Salmonella typhi, Salmonella cholera-suis, Salmonella enteritidis), Clostridium species (e.g., Clostridium perfringens, Clostridium dificile, Clostridium botulinum), Camphlobacter species (e.g., Camphlobacter jejuni, Camphlobacter fetus), Heliobacter species, (e.g., Heliobacter pylori), Aeromonas species (e.g., Aeromonas sobria, Aeromonas hydrophila, Aeromonas caviae), Pleisomonas shigelloides, Yersina enterocolitica, Vibrios species (e.g., Vibrios cholerae, Vibrios parahemolyticus), Klebsiella species, Pseudomonas aeruginosa, and Streptococci. See, e.g., Stein, ed., INTERNAL MEDICINE, 3rd ed., pp 1-13, Little, Brown and Co., Boston, (1990); Evans et al., eds., Bacterial Infections of Humans: Epidemiology and Control, 2d. Ed., pp 239-254, Plenum Medical Book Co., New York (1991); Mandell et al, Principles and Practice of Infectious Diseases, 3d. Ed., Churchill Livingstone, New York (1990); Berkow et al, eds., The Merck Manual, 16th edition, Merck and Co., Rahway, N.J., 1992; Wood et al, FEMS Microbiology Immunology, 76:121-134 (1991); Marrack et al, Science, 248:705-711 (1990), the contents of which references are incorporated entirely herein by reference.

IL18 or IL-18R protein compounds, compositions or combinations of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the IL18 or IL-18R protein composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary but non-limiting protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

IL18 or IL-18R protein compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, IL18 or IL-18R protein compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the IL18 or IL-18R protein compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferrred carrier or excipient materials are carbohydrates (e.g., saccharides and alditols) and buffers (e.g., citrate) or polymeric agents.

Formulations

As noted above, the invention provides for stable formulations, which is preferably a phosphate buffer with saline or a chosen salt, as well as preserved solutions and formulations containing a preservative as well as multi-use preserved formulations suitable for pharmaceutical or veterinary use, comprising at least one IL18 or IL-18R protein in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one IL18 or IL-18R protein with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one IL18 or IL-18R protein, and a second vial comprising an aqueous diluent of prescribed buffer or preservative, wherein said packaging material comprises a label that instructs a patient to reconstitute the at least one IL18 or IL-18R protein in the aqueous diluent to form a solution that can be held over a period of twenty-four hours or greater.

The at least one IL18 or IL-18R protein used in accordance with the present invention can be produced by recombinant means, including from mammalian cell or transgenic preparations, or can be purified from other biological sources, as described herein or as known in the art.

The range of at least one IL18 or IL-18R protein in at least one product of the present invention includes amounts yielding upon reconstitution, if in a wet/dry system, concentrations from about 1.0 ng/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

The range of at least one IL18 or IL-18R protein in at least one product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one IL18 or IL-18R protein and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one IL18 or IL-18R protein and preservative in an aqueous diluent is carried out using conventional dissolution and mixing procedures. To prepare a suitable formulation, for example, a measured amount of at least one IL18 or IL-18R protein in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed formulations can be provided to patients as clear solutions or as dual vials comprising a vial of lyophilized at least one IL18 or IL-18R protein that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2 to about 40° C. and retain the biologically activity of the protein for extended periods of time, thus, allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one IL18 or IL-18R protein in the invention can be prepared by a process that comprises mixing at least one protein in an aqueous diluent. Mixing is carried out using conventional dissolution and mixing procedures. To prepare a suitable diluent, for example, a measured amount of at least one protein in water or buffer is combined in quantities sufficient to provide the protein and optionally a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one IL18 or IL-18R protein that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one IL18 or IL-18R protein that is reconstituted with a second vial containing the aqueous diluent. The clear solution in this case can be up to one liter or even larger in size, providing a large reservoir from which smaller portions of the at least one protein solution can be retrieved one or multiple times for transfer into smaller vials and provided by the pharmacy or clinic to their customers and/or patients.

Recognized devices comprising these single vial systems include those pen-injector devices for delivery of a solution such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com). Recognized devices comprising a dual vial system include those pen-injector systems for reconstituting a lyophilized drug in a cartridge for delivery of the reconstituted solution such as the HumatroPen®.

The products presently claimed include packaging material. The packaging material provides, in addition to the information required by the regulatory agencies, the conditions under which the product can be used. The packaging material of the present invention provides instructions to the patient to reconstitute the at least one IL18 or IL-18R protein in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one IL18 or IL-18R protein and a selected buffer, preferably a phosphate buffer containing saline or a chosen salt. Mixing the at least one protein and buffer in an aqueous diluent is carried out using conventional dissolution and mixing procedures. To prepare a suitable formulation, for example, a measured amount of at least one protein in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one IL18 or IL-18R protein that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one IL18 or IL-18R protein in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention also provides a method for modulating or treating at least one IL18 or IL-18R related disease, in a cell, tissue, organ, animal, or patient, as known in the art or as described herein, using at least one IL-18 or IL-18R protein to mature DCs ex vivo, in vitro or in vivo, according to the present invention.

The present invention also provides a method for modulating or treating at least one IL18 or IL-18R related disease, in a cell, tissue, organ, animal, or patient including, but not limited to, at least one of obesity, an immune related disease, a cardiovascular disease, an infectious disease, a malignant disease or a neurologic disease.

The present invention also provides a method for modulating or treating at least one adult or pediatric immune or inflammation related disease, in a cell, tissue, organ, animal, or patient including, but not limited to, at least one of, or at least one inflammation related to, rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, Crohn's disease, systemic lupus erythematosis, antiphospholipid syndrome, iridocyclitis, uveitis, optic neuritis, idiopathic pulmonary fibrosis, systemic vasculitis, Wegener's granulomatosis, sarcoidosis, orchitis, vasectomy or vasectomy reversal procedures, allergic atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma, hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, type I or type II diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, receptor hypersensitivity reactions, chronic obstructive pulmonary disease (COPD), Graves disease, Raynoud's disease, type B insulin-resistant diabetes, asthma, myasthenia gravis, antibody-meditated cytotoxicity, gene therapy inflammation (e.g., adenovirus, AAV, vaccinia, DNA or RNA, Muloney murine leukemia virus (MMLV) and the like), type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic, idiopathic, Wilson's disease, hemachromatosis, alpha-1-antitrypsin deficiency, diabetic retinopathy, Hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, cd3 therapy, cytokine therapy, chemotherapy, radiation therapy (e.g., including but not limited toasthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

The present invention also provides a method for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, restenosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflammation response, chaotic or multifocal atrial tachycardia, regular narrow QRS tachycardia, specific arrythmias, ventricular fibrillation, His bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occulsion of the abdominal aorta and its branches, peripheral vascular disorders, occulsive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like. Such a method can optionally comprise administering an effective amount of a composition or pharmaceutical composition comprising at least one IL18 or IL-18R protein to a cell, tissue, organ, animal or patient in need of such modulation, treatment or therapy.

The present invention also provides a method for modulating or treating at least one infectious disease in a cell, tissue, organ, animal or patient, including, but not limited to, at least one of: acute or chronic infection, acute and chronic parasitic or infectious processes, including bacterial, viral and fungal infections, HIV infection, HIV neuropathy, meningitis, hepatitis (A, B or C, or the like), septic arthritis, peritonitis, pneumonia, epiglottitis, e. coli 0157:h7, hemolytic uremic syndrome, thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis, epidydimitis, legionella, lyme disease, influenza a, epstein-barr virus, vital-associated hemaphagocytic syndrome, vital encephalitis, aseptic meningitis, and the like. Such toxins can be, but are not limited to, purified or recombinant toxin or toxin fragment comprising at least one functional cytotoxic domain of toxin, e.g., selected from at least one of diphtheria toxin, a venom toxin, a viral toxin or a bacterial toxin. The term toxin also includes both endotoxins and exotoxins produced by any naturally occurring, mutant or recombinant bacteria or viruses which may cause any pathological condition in humans and other mammals, including toxin shock, which can result in death. Such toxins may include, but are not limited to, enterotoxigenic E. coli heat-labile enterotoxin (LT), heat-stable enterotoxin (ST), Shigella cytotoxin, Aeromonas enterotoxins, toxic shock syndrome toxin-1 (TSST-1), Staphylococcal enterotoxin A (SEA), B (SEB), or C (SEC), Streptococcal enterotoxins anthrax endotoxin, and the like. Such bacteria include, but are not limited to, gram negative or gram positive bactieria, Bacillus, E. coli, Streptococcus, Staphlococcus, Shigella, Salmonella, Clostridium, Camphbacter, Heliobacter, Aeromonas, Enteroccis, Pseudomonas, and the like, such as but not limited to, strains of a species of enterotoxigenic E. coli (ETEC), enterohemorrhagic E. coli (e.g., strains of serotype 0157:H7), Staphylococcus species (e.g., Staphylococcus aureus, Staphylococcus pyogenes), Shigella species (e.g., Shigella dysenteriae, Shigella flexneri, Shigella boydii, and Shigella sonnei), Salmonella species (e.g., Salmonella typhi, Salmonella cholera-suis, Salmonella enteritidis), Clostridium species (e.g., Clostridium perfringens, Clostridium dificile, Clostridium botulinum), Camphlobacter species (e.g., Camphlobacter jejuni, Camphlobacter fetus), Heliobacter species, (e.g., Heliobacter pylori), Aeromonas species (e.g., Aeromonas sobria, Aeromonas hydrophila, Aeromonas caviae), Pleisomonas shigelloides, Yersina enterocolitica, Vibrios species (e.g., Vibrios cholerae, Vibrios parahemolyticus), Klebsiella species, Pseudomonas aeruginosa, and Streptococci. See, e.g., Stein, ed., INTERNAL MEDICINE, 3rd ed., pp 1-13, Little, Brown and Co., Boston, (1990); Evans et al., eds., Bacterial Infections of Humans: Epidemiology and Control, 2d. Ed., pp 239-254, Plenum Medical Book Co., New York (1991); Mandell et al, Principles and Practice of Infectious Diseases, 3d. Ed., Churchill Livingstone, New York (1990); Berkow et al, eds., The Merck Manual, 16th edition, Merck and Co., Rahway, N.J., 1992; Wood et al, FEMS Microbiology Immunology, 76:121-134 (1991); Marrack et al, Science, 248:705-711 (1990), the contents of which references are incorporated entirely herein by reference. Such a method can optionally comprise administering an effective amount of a composition or pharmaceutical composition comprising at least one IL18 or IL-18R protein to a cell, tissue, organ, animal or patient in need of such modulation, treatment or therapy.

The present invention also provides a method for modulating or treating at least one malignant disease in a cell, tissue, organ, animal or patient, including, but not limited to, at least one of: leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome, hypercalcemia of malignancy, solid tumors, CD-46 related tumors, adenocarcinomas, sarcomas, malignant melanoma, hemangioma, metastatic disease, cancer related bone resorption, cancer related bone pain, and the like. Such a method can optionally comprise administering an effective amount of a composition or pharmaceutical composition comprising at least one IL18 or IL-18R protein to a cell, tissue, organ, animal or patient in need of such modulation, treatment or therapy.

The present invention also provides a method for modulating or treating at least one neurologic disease in a cell, tissue, organ, animal or patient, including, but not limited to, at least one of: neurodegenerative diseases, multiple sclerosis, migraine headache, AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi.system disorder); demyelinating core disorders, such as multiple sclerosis, acute transverse myelitis; and disorders of the motor unit such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, and the like. Such a method can optionally comprise administering an effective amount of a composition or pharmaceutical composition comprising at least one IL18 or IL-18R protein to a cell, tissue, organ, animal or patient in need of such modulation, treatment or therapy. See, e.g., the Merck Manual, 16^{th} Edition, Merck & Company, Rahway, N.J. (1992)

Any method of the present invention can comprise administering an effective amount of a composition or pharmaceutical composition comprising at least one IL18 or IL-18R protein to a cell, tissue, organ, animal or patient in need of such modulation, treatment or therapy that includes maturing DCs. Such a method can optionally further comprise co-administration or combination therapy for treating such diseases, wherein the administering of said at least one IL18 or IL-18R protein, specified portion or variant thereof, further comprises administering, before concurrently, and/or after, at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

TNF antagonists suitable for compositions, combination therapy, co-administration, devices and/or methods of the present invention (further comprising at least one anti body, specified portion and variant thereof, of the present invention), include, but are not limited to, TNF proteins, antigen-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g, pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody,” “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes TNF antibodies, antigen-binding fragments thereof, and specified mutants or domains thereof that bind specifically to TNFα. A suitable TNF anttibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric antibody cA2 consists of the antigen binding variable region of the high-affinity neutralizing mouse human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 (cA2) neutralizes the cytotoxic effect of both natural and recombinant human TNFα in a dose dependent manner. From binding assays of chimeric antibody cA2 and recombinant human TNFα, the affinity constant of chimeric antibody cA2 was calculated to be 1.04×¹⁰M⁻¹. Preferred methods for determining monoclonal antibody specificity and affinity by competitive inhibition can be found in Harlow, et al., antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992-2000); Kozbor et al., Immunol. Today, 4:72-79 (1983); Ausubel et al., eds. Current Protocols in Molecular Biology, Wiley Interscience, New York (1987-2000); and Muller, Meth. Enzymol., 92:589-601 (1983), which references are entirely incorporated herein by reference.

In a particular embodiment, murine monoclonal antibody A2 is produced by a cell line designated c134A. Chimeric antibody cA2 is produced by a cell line designated c168A.

Additional examples of monoclonal TNF antibodies that can be used in the present invention are described in the art (see, e.g., U.S. Pat. No. 5,231,024; Möller, A. et al., Cytokine 2(3):162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

TNF Receptor Molecules

Preferred TNF receptor molecules useful in the present invention are those that bind TNFα with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, can contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNF□ with high affinity and possess low immunogenicity). A functional equivalent of TNF receptor molecule also includes modified TNF receptor molecules that functionally resemble TNF receptor molecules that can be used in the present invention (e.g., bind TNF□ with high affinity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2000).

Cytokines include any known cytokine. See, e.g., CopewithCytokines.com. Cytokine antagonists include, but are not limited to, any antibody, fragment or mimetic, any soluble receptor, fragment or mimetic, any small molecule antagonist, or any combination thereof.

Therapeutic Treatments. Any method of the present invention can comprise a method for treating a IL18 or IL-18R mediated disorder or disease through the maturation of DC, comprising administering an effective amount of a composition or pharmaceutical composition comprising at least one IL18 or IL-18R protein to a cell, tissue, organ, animal or patient in need of such modulation, treatment or therapy. Such a method can optionally further comprise co-administration or combination therapy for treating such disorders or diseases, wherein the administering of said at least one IL8 or IL-18R protein, further comprises administering, before concurrently, and/or after, at least one selected from at least one at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist.

Protein Dosing

Typically, treatment of pathologic conditions is effected by administering an effective amount or dosage of at least one IL18 or IL-18R protein composition for maturing DCs, that total, on average, a range from at least about 0.001 ng to 500 milligrams of at least one IL18 or IL-18R protein per kilogram of patient or DCs or blood per dose, and preferably from at least about 0.1 ng to 100 milligrams antibody/kilogram of patient or DCs or blood per single or multiple administration, depending upon the specific activity of contained in the composition. Alternatively, the effective concentration can comprise 0.001 ng-0.05 mg/ml concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses of at least one protein can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 micrograms or milligrams/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 ng μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 μg to 100 milligrams per kilogram of body weight. Ordinarily 0.0001 to 50, and preferably 0.001 to 10 milligrams per kilogram per administration or in sustained release form is effective to obtain desired results.

As a non-limiting example, treatment of humans or animals can be provided as a one-time or periodic dosage of at least one protein of the present invention 0.1 to 100 μg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90, 100, 200, 300, 400, 500, 600, 700, 800, 900, 1000, 2000 or 3000 μg/kg, per day, or 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.00001 milligram to about 500 milligrams of active ingredient per unit or container. In these pharmaceutical compositions the active ingredient will ordinarily be present in an amount of about 0.5-99.999% by weight based on the total weight of the composition.

Typically, treatment of pathologic conditions is effected by administering an effective amount or dosage of at least one IL18 or IL-18R protein composition that total, on average, a range from at least about 0.00001 to 500 milligrams of at least one IL18 or IL-18R protein per kilogram of patient per dose, and preferably from at least about 0.0001 to 100 milligrams protein/kilogram of DC, tissue, or patient per single or multiple administration, depending upon the specific activity of contained in the composition. Alternatively, the effective serum concentration can comprise 0.0001-500 μg/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Dosage forms (composition) suitable for internal administration generally contain from about 0.1 milligram to about 500 milligrams of active ingredient per unit or container. In these pharmaceutical compositions the active ingredient will ordinarily be present in an amount of about 0.5-99.999% by weight based on the total weight of the composition.

Administration

For parenteral administration, the protein can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes of can be used according to the present invention for administering pharmaceutically effective amounts of at least one IL18 or IL-18R protein according to the present invention. While pulmonary administration is used in the following description, other modes of administration can be used according to the present invention with suitable results.

IL18 or IL-18R protein of the present invention can be delivered in a carrier, as a solution, emulsion, colloid, or suspension, or as a dry powder, using any of a variety of devices and methods suitable for administration by inhalation or other modes described here within or known in the art.

Parenteral Formulations and Administration

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aquous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of at least one IL18 or IL-18R protein by parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal means. At least one IL18 or IL-18R protein composition can be prepared for use for parenteral (subcutaneous, intramuscular or intravenous) or any other administration particularly in the form of liquid solutions or suspensions; for use in vaginal or rectal administration particularly in semisolid forms such as, but not limited to, creams and suppositories; for buccal, or sublingual administration such as, but not limited to, in the form of tablets or capsules; or intranasally such as, but not limited to, the form of powders, nasal drops or aerosols or certain agents; or transdermally such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

Pulmonary/Nasal Administration

For pulmonary administration, preferably at least one IL18 or IL-18R protein composition is delivered in a particle size effective for reaching the lower airways of the lung or sinuses. According to the invention, at least one IL18 or IL-18R protein can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of antibodies are also known in the art. All such devices can use of formulations suitable for the administration for the dispensing of protein in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention. Preferably, a composition comprising at least one IL18 or IL-18R protein is delivered by a dry powder inhaler or a sprayer. There are a several desirable features of an inhalation device for administering at least one protein of the present invention. For example, delivery by the inhalation device is advantageously reliable, reproducible, and accurate. The inhalation device can optionally deliver small dry particles, e.g. less than about 10 μm, preferably about 1-5 μm, for good respirability.

### Administration of IL18 or IL-18R protein Compositions as a Spray

A spray including IL18 or IL-18R protein composition can be produced by forcing a suspension or solution of at least one IL18 or IL-18R protein through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one IL18 or IL-18R protein composition delivered by a sprayer have a particle size less than about 10 μm, preferably in the range of about 1 μm to about 5 μm, and most preferably about 2 μm to about 3 μm.

Formulations of at least one IL18 or IL-18R protein composition suitable for use with a sprayer typically include protein compositions in an aqueous solution at a concentration of about 0.0000001 mg to about 1000 mg of at least one IL18 or IL-18R protein composition per ml of solution or mg/gm, or any range or value therein, e.g., but not lmited to, 0.1, 0.2., 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 ng or μg or mg/ml or ng or μg or mg/gm. The formulation can include agents such as an excipient, a buffer, an isotonicity agent, a preservative, a surfactant, and, preferably, zinc. The formulation can also include an excipient or agent for stabilization of the protein composition, such as a buffer, a reducing agent, a bulk protein, or a carbohydrate. Bulk proteins useful in formulating protein compositions include albumin, protamine, or the like. Typical carbohydrates useful in formulating protein compositions include sucrose, mannitol, lactose, trehalose, glucose, or the like. The protein composition formulation can also include a surfactant, which can reduce or prevent surface-induced aggregation of the protein composition caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein such as IL18 or IL-18R antibodies, or specified portions or variants, can also be included in the formulation.

### Administration of IL18 or IL-18R Protein Compositions by a Nebulizer

Such a protein composition can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of protein composition through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of protein composition either directly or through a coupling fluid, creating an aerosol including the protein composition. Advantageously, particles of protein composition delivered by a nebulizer have a particle size less than about 10 μm, preferably in the range of about 1 μm to about 5 μm, and most preferably about 2 μm to about 3 μm.

Formulations of at least one IL18 or IL-18R protein suitable for use with a nebulizer, either jet or ultrasonic, typically include a concentration of about 0.1 mg to about 100 mg of at least one IL18 or IL-18R protein protein per ml of solution. The formulation can include agents such as an excipient, a buffer, an isotonicity agent, a preservative, a surfactant, and, preferably, zinc. The formulation can also include an excipient or agent for stabilization of the at least one IL18 or IL-18R protein composition, such as a buffer, a reducing agent, a bulk protein, or a carbohydrate. Bulk proteins useful in formulating at least one IL18 or IL-18R protein compositions include albumin, protamine, or the like. Typical carbohydrates useful in formulating at least one IL18 or IL-18R protein include sucrose, mannitol, lactose, trehalose, glucose, or the like. The at least one IL18 or IL-18R protein formulation can also include a surfactant, which can reduce or prevent surface-induced aggregation of the at least one IL18 or IL-18R protein caused by atomization of the solution in forming an aerosol. Various conventional surfactants can be employed, such as polyoxyethylene fatty acid esters and alcohols, and polyoxyethylene sorbital fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein can also be included in the formulation.

Administration of IL18 OR IL-18R Protein Compositions By A Metered Dose Inhaler

In a metered dose inhaler (MDI), a propellant, at least one IL18 or IL-18R protein, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of protein composition produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of at least one IL18 or IL-18R protein for use with a metered-dose inhaler device will generally include a finely divided powder containing at least one IL18 or IL-18R protein as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably the propellant is a hydrofluorocarbon. The surfactant can be chosen to stabilize the at least one IL18 or IL-18R protein as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein such as protein can also be included in the formulation.

One of ordinary skill in the art will recognize that the methods of the current invention can be achieved by pulmonary administration of at least one IL18 or IL-18R protein compositions via devices not described herein.

Oral Formulations and Administration

Formulations for oral rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 and U.S. Pat. No. 5,5,871,753 are used to deliver biologically active agents orally are known in the art.

Mucosal Formulations and Administration

For absorption through mucosal surfaces, compositions and methods of administering at least one IL8 or IL-18R protein include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. Nos. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like (U.S. Pat. Nos. 5,849,695).

Transdermal Formulations and Administration

For transdermal administration, the at least one IL18 or IL-18R protein is encapsulated in a delivery device such as a liposome or polymeric nanoparticles, microparticle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. Nos. 5,814,599).

Prolonged Administration and Formulations

It can be sometimes desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds of the present invention or, preferably, a relatively insoluble salt such as those just described, can be formulated in a gel, for example, an aluminum monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. Nos. 5,770,222 and “Sustained and Controlled Release Drug Delivery Systems”, J. R. Robinson ed., Marcel Dekker, Inc., N.Y., 1978).

Having generally described the invention, the same will be more readily understood by reference to the following examples, which are provided by way of illustration and are not intended as limiting.

Cloning and Expression of IL18 OR IL-18R Protein in Mammalian Cells

A typical mammalian expression vector contains at least one promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded protein or protein, e.g., as a desired portion of at least one of SEQ ID NOS:1-2. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are used for the production of antibodies or proteins of the present invention.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

Cloning and Expression in CHO Cells

The vector pC4 is used for the expression of IL18 or IL-18R protein, e.g., using a coding sequence for at least one of SEQ ID NOS:1-2. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains coding DNA for expressing the gene of interest under control of the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the IL18 or IL-18R in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It can be advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the desired IL18 or IL-18R protein is used, e.g., DNA or RNA coding for at least one of SEQ ID NOS:1-2, corresponding to at least one portion of at least one IL18 or IL-18R protein protein of the present invention, according to known method steps.

The isolated encoding DNA and the dephosphorylated vector are then ligated with T4 DNA ligase. E. coli HB 101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

Generation of IL-18 Muteins

IL-1 and IL-1 receptor are structurally homologous to IL-18 and IL-18 respectively. Using the crystal structure of IL-1β with its receptor from the Brookhaven Data Bank, a model of IL-18/IL-18 receptor was constructed. Amino acids were electronically mutated, from IL-1β and of IL-1β receptor to the corresponding amino acids in human IL-18 and IL-18 receptor. Additions and deletions were handled by performing loop searches anchored at residues appearing on both molecules. Loops were examined for bond angles, interaction of backbone and side chains and rationality of position. The resulting structure was subjected to minimization and dynamics. Individual amino acids in IL-18 were examined and their interaction with the IL-18 receptor evaluated. Based on the model, rational substitutions were suggested that would either retain or alter IL-18 activity. The substitutions defined here are not meant to be the only substitutions possible or to limit the utility of this model. The muteins identified using this model are useful as IL-18 agonists, IL-18 antagonists, for raising anti-IL-18 antibodies and for substitution for IL-18 in assays, models, and other IL-18 functions.

Using the crystal structure of IL-1 with its receptor, the sequence of IL-1 was aligned with IL18.

| | |
|---|---|
| | 1 |
| IL-1 | --APVRSLNC TLRDSQQKSL VM---SGPYE LKALHLQGQD MEQQVVFSMS |
| IL-18 | YFGKLESKLS VIRNLNDQVL FIDQGNRPLF EDMTDSDCRD NAPRTIFIIS |
| | 51 |
| IL-1 | FVQGEESNDK IPVALGLKEK NLYLSCVLKD DKPTLQLESV DPKNYPKKKM |
| IL-18 | MYKDSQPRGM AVTISVKCEK ISTLSC---- ENKIISFKEM NPPDNIKDTK |
| | 101 |
| IL-1 | EKRFVFNKI- -EINNKLEFE SAQFPNWYIS TSQAENMPVF LGGTKGGQDI |
| IL-18 | SDIIFFQRSV PGHDNKMQFE SSSYEGYFLA CEKERDLFKL I--LKKEDEL |
| | 151 |
| IL-1 | TDFTMQFVSS --- |
| IL-18 | GDRSIMFTVQ NED |

Initial amino acid numbering refers to the positions in IL-1 and the IL-1 receptor. Once the IL-18/IL-18 receptor structure was complete, the structure was renumbered to be consistent with IL-18/IL-18 receptor numbering. The amino acids in IL-1 were electronically mutated to the IL-18 sequence. Additions or deletions were ignored at this point.

The sequence of the IL-1 receptor was aligned with the sequence for the IL-18 receptor.

| | |
|---|---|
| | 1 |
| IL-1r | --CKEREEKI ILVSSANEID VRPCPLNPNE HKGTITWYK- --DDSKTPVS |
| IL-18r | ESCTSRPHIT VVEGEPFYLK HCSCSLAHEI ETTTKSWYKS SGSQEHVELN |
| | 51 |
| IL-1r | TEQASRIHQH KEKLWFVPAK VEDSGHYYCV VRNSSYCLRI KISAKFVENE |
| IL-18r | PRSSSRIALH DCVLEFWPVE LNDTGSYFFQ MKN--YTQKW KLN--VIRRN |
| | 101 |
| IL-1r | PNLCYNAQAI FKQKLPVAGD GGLVC--PYM EFFKNENNEL PKLQWYKDCK |
| I-18r | KHSCFTERQV TSKIVEVKKF FQITCENSYY QTLVNSTS-- ----LYKNCK |
| | 151 |
| IL-1r | PLLLDNIHFS GVKDRLIVMN VAEKHRGNYT CHASYTYLGK QYPITRVIEF |
| IL-18r | KLLLEN---- -NKNPTIKKN AEFEDQGYYS CVHFLHHNGK LFNITKTFNI |
| | 201 |
| IL-1r | ITLEENKPTR PVIVSPANET MEVDLGSQIQ LICNVTGQLS DIAYWKWNGS |
| IL-18r | TIVEDRSNIV PVLLGPKLNH VAVELGKNVR LNCSALLNEE DVIYWMFGEE |
| | 251 |
| IL-1r | VIDEDDPVLG EDY-YSVENP ANKRRSTLIT VLNISEIESR FYKHPFTCFA |
| IL-18r | --NGSDPNIH EEKEMRIMTP EGKWHAS--K VLRIENIGES NLNVLYNCTV |
| | 301 |
| IL-1r | KNTHGIDAAY IQLIYPVT |
| IL-18r | ASTGGTDTKS FILVRKA- |

Individual amino acids in the IL-1 receptor were electronically mutated and the potential effect of the changes on the interaction with IL-18 was evaluated. An examination of the structure amino acid-by-amino acid led to the following observations:

Cys¹ forms a disulfide bond with Cys⁸². In the IL-18 receptor the equivalent of Cys⁸² is absent.

The change of Val¹¹ to Glu creates possible hydrogen bonding with Arg³⁴ in IL-18, an amino acid different from that in IL-1. Arg²⁰ to Cys gives an apparently unpaired cysteine but Glu⁵⁷ becomes a cysteine as well and is in the immediate vicinity. This becomes a new disulfide bond but geometry needs to be adjusted. Pro²⁶ to His adds an aromatic residue that will interact with the two new aromatics in IL-18, Phe²⁵ and Phe¹³¹. The loop from 45-51 needs to be redone because of a bad bend resulting from the new proline at 46. A disulfide is created between 20 and 57. This is a very long bond (14.257 angstroms) and some geometric correction is necessary. Cys⁷⁴ to Phe removes the disulfide bond with 22. Cys⁸² to Thr removes the disulfide bond with Cys¹. Pro¹¹¹ to Glu gives hydrogen bonding potential with IL-18 Arg¹¹ and Lys¹⁰⁹ (both unchanged from IL-1). Gln¹⁰⁸ to Lys gives hydrogen bonding potential with the Gln¹⁵ to Asp IL-18 mutation. Asn¹⁹⁹ to Arg creates the possibility of π-π interactions with Phe¹⁵⁰ in IL-18. Tyr²⁵⁶ to Lys and Ser²⁵⁸ to Arg give possible hydrogen bonding with IL-18 Glu⁴ that was an Arg. The regions where additions and/or deletions in the two sequences were present were identified. There are 3 regions where the additions are involved in contact between IL-18 and the receptor. These are underlined on the alignment sequences above.

Insertions and Deletions from the Sequences

The sequence VLKD in IL-1 is an external loop with no receptor contact. This sequence is deleted in IL-18. A loop search was done using CyS⁷¹ and Ile⁸⁰ as anchor points and searching for ENKI. This deleted the four amino acids and created a new loop. Of the loops identified, 1 QBA:Arg825 gave a good fit and positioned the side chains such that the Glu hydrogen binds with the side chain of Lys⁸³ and Tyr¹¹⁷ and Tyr¹²⁰ can form a π-π interaction. To remove the Gly¹³⁵⁻¹³⁶ in IL-1, a loop search was done, anchoring at Ile¹³⁴ and Gly¹⁴⁴ and searching for the loop LKKEDE. The loop 1AHJ:D/Glu134 that placed all hydrophilic residues on the surface was inserted. The sequence NED was added to the C-terminus in a trans configuration. This allowed hydrogen bonding with the Glu and Arg²⁵⁸ of the receptor. There were two deletions in the IL-18 receptor close together (SS and AK). Both of these were done together since they are part of a long beta structure. The loop was anchored at Met⁷⁶ and Val⁹¹ and the sequence KNYTQKWKLN was searched. There is only one loop that gives trans amide bonds, the tyrosine giving π-π interactions with Arg² and the Trp giving π-π interactions with His⁶, 1CHM:B/Met253. This was inserted, the side chains relaxed. To remove IHFSG from the IL-1 receptor, a loop search anchoring at Leu¹⁴⁵ and Ile¹⁶⁰ and searching for LLENNKNKPT was done. The loop 1LPB:B/Phe72 was inserted. To remove ELPKLQ from the IL-1 receptor, a loop search using Tyr¹²³ and Leu¹³⁸ as anchors was done, searching for QTLVNSTS. The loop 1FEC:B/Tyr182 was inserted. The sequence EGKWHAS—in IL-18 receptor was changed to—EGKWHAS and a loop search was done to modify the hairpin turn by removing EG. The anchor residues were Thr²⁶¹ and Lys²⁶⁷ and the search was for PEG. The loop 2FB4:H/Ser¹³⁵ was inserted. To remove VI from the IL-1 receptor, a search for FGEEN was done, anchoring at Met²³⁹ and Gly²⁴⁷. The loop 1BRB:E/Arg67 was inserted. This region contacts the IL-18.

EEKEMRI needed the underlined E added. This is an IL-18 contact residue. This was an opportunity to remove some of the interactions between Trp²⁶⁸ and Ile²⁵⁹. A loop search was done using Thr²⁶¹ and Glu²⁵⁴ as anchor points and searching the sequence KEMRIM. The loop 1IND:L/Trp98 was inserted. A search for the sequence SSGSQE was done, anchoring at Lys³⁷ and His⁴¹. The loop 1SLT:B/Asn61 was inserted. The introduction of the VP into IL-18 was not simple. Based on the alignment, this is on the side of a loop, the tip of which contacts the receptor. The Asp probably hydrogen bonds with Lys¹¹⁴ of the receptor. A loop search, anchoring at Arg¹⁰³ and Asn¹⁰⁸ and searching the sequence SVPGHD, was done. The loop 1TDT:B/Thr212 was inserted.

For DQG in IL-18, a search for IDQGNRP, anchoring at Phe¹⁹ and Leu²⁴, was done. The loop 1PYS:B/Leu730 was inserted. The disulfide pairing in the IL-18 receptor was adjusted. From examination of the model, it was highly unlikely that cysteines 20 and 22 pair up. The most likely pairing was 22 with 57 and 20 with 1. This necessitated repositioning the loop of 73 to 82 to allow bringing the chain from 1 to 7 close enough to form the disulfide bond between 1 and 20. The loop 72-83 was deleted and the 1-7 sequence repositioned for disulfide bond formation by manipulating bond angles.

The torsional angle between Arg⁴ and Pro⁵ was modified from 149 degrees to 209 degrees. This placed the cysteine sulfurs 7.2 angstroms apart but with nothing in between. The distance between the sulfurs in cysteines 20 and 57 was 12 angstroms but the side chain on 20 was pointed in the wrong direction. Amino acids 1-4 of the receptor were manually positioned them so that they filled the gap around the Cys²⁰ and had the two cysteines close enough to form the disulfide bond. This was merged with the structure. Amino acids 1-4 were deleted from the receptor and a bond was formed between the new 4 and old 5. A loop search was then done using Thr² and Val¹⁰ as anchors and searching for SRPHITF. The loop from 1EZM:Phe54 was inserted. The loop between 72 and 83 was replaced. Using anchors at Asp⁶⁸ and Leu⁸⁵, a search for TGSYFFQMKNYTQKWK was done. The loop from 2CAS:Gly412 was inserted.

The resulting structure was refined as follows: The structure was minimize using steepest descent, 100 cycles, 8 angstroms for non-bonded cutoff, 100 dielectric, Tripos force field, kollman-all charges. A dynamics run was done (100 fs, random, NPT, 300 deg, 5 atm) followed by minimization (steepest descent, 100 cycles, 8 angstroms for non-bonded cutoff, 100 dielectric, Tripos force field, kollman-all charges). A final minimization was done (conjugate gradient, 100 cycles, 8 angstroms for non-bonded cutoff, 100 dielectric, Tripos force field, kollman-all charges). The resulting structure had inverted the chirality of Tyr¹. Tyr¹-Phe² was repositioned and local minimization done (conjugate gradient, 100 cycles, 8 angstroms for non-bonded cutoff, 100 dielectric, Tripos force field, kollman-all charges). The resulting model was examined amino acid-by-amino acid to determine the effect of potential amino acid substitutions on IL-18/IL-18 receptor interactions. The following observations were made:

Tyr¹-Phe² These residues probably interact with the receptor and changing them would affect binding. Interaction is peripheral (at the edge of the receptor-ligand interface). I believe these residues to be important. Substitution by non-aromatic residues could reduce affinity. Lys⁴ may interact with Glu²⁴¹ and is peripheral. Leu⁵ is internal and could be substituted by valine. Glu⁶ probably interacts with Arg²⁴⁵. Lys⁸ interacts with the receptor and is critical. Ser¹⁰ could be replaced by Thr. Val¹¹ could be replaced by Ile. Ile¹² could be replaced by Val. Arg¹³ is probably a receptor contact residue. Leu¹⁵ may interact peripherally. Asp¹⁷ is a receptor contact residue and could be replaced by Asn. Gln¹⁸ may be a receptor contact residue. Leu²⁰ could be replaced by Val or Ile. Phe²¹ could be replaced by Tyr. Ile²² could be replaced by Val. Arg²⁷ is a peripheral receptor contact residue Leu²⁹ could be replaced by Val. Phe³⁰ is a residue contact residue that could be replaced by Tyr. Asp³⁵ is a receptor contact residue. DCRD (37-40) are receptor contact residues. Arg³⁹ is a receptor contact residue. Long shot, but it may be able to be substituted with a Trp. Ala⁴² is involved in a beta turn with Pro⁴³. Ala⁴² could be substituted with a Ser. Thr⁴⁵ could be replaced with Ser. Ile⁴⁶ could be replaced with Val. Phe⁴⁷ could be replaced with Tyr and it would add hydrogen bonding to Lys¹³⁵. Ser⁵⁰ could be replaced by Arg or Asn. Met⁵¹ is a possible receptor contact residue. Tyr⁵² could be replaced with Phe. Lys⁵³ is a critical receptor contact residue. Gin⁵⁶ is a receptor contact residue. A possible substitution would be Glu. Arg⁵⁸ is a receptor contact residue. Val⁶² is a receptor contact residue. Thr⁶³ could be replaced by Ala. Ile⁶⁴/Val⁶⁶ could be simultaneously replaced with Val⁶⁴/Ile⁶⁶. Glu⁶⁹ could be replaced with Gln, Asp or Asn. Ser⁷² could be replaced with Thr. Glu⁷⁷ could be replaced with Asp or Gln. Lys⁷⁹ could be replaced by Arg. Ser⁸² could be replaced with Thr. Glu⁷⁷ could be replaced with Asp. Met⁸⁶ could be replaced by Val, Gln or Asn. Asn⁸⁷ could be replaced with Gln. Pro⁸⁸ could be replaced with Ser. Ile⁹² could be replaced with Val. Asp⁹⁴ and Thr⁹⁵ are receptor contact residues. Asp⁹⁸ could be replaced with Glu or Asn. Phe¹⁰¹ could be replaced with Tyr. Arg¹⁰⁴ is receptor binding and critical. GHDN (108-111) are possible receptor contact residues. Gln¹¹⁴ could be replaced by Asn. Ser¹¹⁸ could be replaced by Thr. Tyr¹²⁰ could be replaced by Phe. Glu¹²¹ could be replaced by Asp. Tyr¹²³ could be replaced by Phe. Phe¹²⁴ could be replaced by Tyr. Ala¹²⁶ could be replaced by Thr. Lys¹²⁹ is a receptor contact residue. Glu¹³⁰ is a possible receptor contact residue. Arg¹³¹ is a receptor contact residue and critical.

Asp¹³² is a receptor contact residue and critical. Leu¹³³ and Phe¹³⁴ are receptor contact residues and critical. Phe could be replaced by Tyr. Glu¹⁴¹ could be replaced by Lys or Asp. Ser¹⁴⁸ is a possible receptor contact residue. Simultaneous substitution of Asp¹¹⁰ by Arg and Ser¹⁴⁸ by Phe could increase binding of IL-18 to its receptor. Met¹⁵⁰ is a receptor contact residue. Phe¹⁵¹ is receptor contact and critical. Gln¹⁵⁴ could be replaced by Asn. Asn¹⁵⁵ could be replaced by Glu or Ser. Glu¹⁵⁶ could be replaced by Asp or Gln. Asp¹⁵⁷ could be replaced by Glu or Asn. A table was prepared in which the side chain and total amino acid surface exposure was calculated.

Residues that could be substituted were identified. Receptor binding residues were identified and a judgement was made as to whether they were on the periphery of the interface between IL-18 and the receptor. These would presumably be less sensitive to substitution. To create agonists, non-receptor contact amino acids could be substituted. To create antagonists, receptor contact residues could be substituted. To create an antigen for raising antibodies, non-surface exposed amino acids could be substituted. To create an antigen for raising neutralizing antibodies, receptor contact residues should be kept intact and both surface and non-surface exposed amino acids could be substituted. To avoid immunogenicity issues, surface amino acid substitutions should be avoided.

Changes in non-surface exposed residues that could be made that would result in the high probability of retention of IL-18 activity with no changes in immunogenicity are:
Thr¹⁰ for Ser¹⁰
Val¹² for Ile¹²
Ser⁴⁵ for Thr⁴⁵
Tyr⁴⁷ for Phe⁴⁷
Phe⁵² for Tyr⁵²
Val⁶⁴ for Ile⁶⁴
Tyr¹⁰¹ for Phe¹⁰¹

These compounds would be useful as IL-18 agonists, for raising anti-IL-18 antibodies, for assays for IL-18 or IL-18 binding proteins and for preparation of affinity columns for the purification of IL-18 binding proteins.

Changes in amino acids with a low percentage of surface exposure that could be made that would result in the high probability of retention of IL-18 activity with possible changes in immunogenicity are:
Val⁵ for Leu⁵
Val²⁰ for Leu²⁰
Ile²⁰ for Leu²⁰
Tyr²¹ for Phe²¹
Val²² for Ile²²
Ile⁶⁶ for Val⁶⁶
Thr⁷² for Ser⁷²
Phe¹⁴⁸ for Ser¹⁴⁸

These compounds would be useful as IL-18 agonists, for raising anti-IL-18 antibodies, for assays for I-18 or IL-18 binding proteins and for preparation of affinity columns for the purification of IL-18 binding proteins.

Changes that could be made in amino acids involved in receptor contact that would result in alteration of IL-18 activity by either increasing or decreasing binding of the IL-18 analog to the IL-18 receptor are:
Glu⁴ for Lys⁴
Ile⁶ for Glu⁶
Asp⁸ for Lys⁸
Ile¹³ for Arg¹³
Arg¹⁵ for Leu¹⁵
Lys¹⁷ for Asp¹⁷
Lys²⁷ for Arg²⁷
Ala³⁰ for Phe³⁰
Lys³⁵ for Asp³⁵
Phe³⁷ for Asp³⁷
Glu³⁸ for Cys³⁸
Ala³⁹ for Arg³⁹
Trp⁴⁰ for Asp⁴⁰
Glu⁵¹ for Met⁵¹
Gly⁵³ for Lys⁵³
Ile⁵⁶ for Gln⁵⁶
Ala⁵⁸ for Arg⁵⁸
Lys⁶² for Val⁶²
Lys⁹⁴ for Asp⁹⁴
Phe⁹⁵ for Thr⁹⁵
Leu¹⁰⁴ for Arg¹⁰⁴
Ile¹⁰⁸ for Gly¹⁰⁸
Lys¹¹¹ for Asn¹¹¹
Phe¹²⁹ for Lys¹²⁹
Asp¹³¹ for Arg¹³¹
Leu¹³² for Asp¹³²
Glu¹³³ for Leu¹³³
Ala¹³⁴ for Phe¹³⁴
Thr¹⁵⁰ for Met¹⁵⁰
Ser¹⁵¹ for Phe¹⁵¹

Depending on the alteration of receptor binding or receptor activity, these compounds would be useful as IL-18 agonists or antagonists, for preparation of antibodies against IL-18, in assays for IL-18 or IL-18 binding proteins and the preparation of affinity columns for the purification of IL-18 binding proteins.

Advantages:

The model described herein has as the advantage of allowing for predicting the effect of changing amino acids in IL-18 and allowing for the rationale design of new and potentially useful IL-18 muteins that do not exist in nature.

### Monocyte-Derived DC Maturation: Up-Regulation Type-1 and Type-2 Cytokine in IL-18 Stimulated KG-1 Cells

IL-18 was first described as an IFNγ inducing factor but it was later found that IL-18 can also induce the type 2 cytokines IL-4 and IL-13 by T cells, NK cells, mast cells and basophils. IL-13 is a Th2 derived cytokine that shares a variety of biological functions with IL-4, such as inducing B cell proliferation, differentiation and immunoglobulin production and inhibition of the production of inflammation cytokine by monocytes. IL-13 can be produced by T cell, mast cell and macrophages. IL-13 appears to be involved in functional maturation of human peripheral blood monocyte-derived DC, however, the cytokine profile expressed by DC is dependent on cell subtype and mode of activation.

We have surprisingly and unexpectedly found that IL-18 can directly up-regulate IL-13 gene, protein and IL-13 receptor gene expression on a myelomonocytic cell line, KG-1 cells. Our data show that human DCs treated with IL-18 increased IL-13 production by allologous lymphocytes (data not shown). It has been showed in human systems, that lymphoid DCs generate type 2 response, while myeloid DCs genereate a type 1 response. We found that KG-1 cells (CD8α negative cells, data not shown) generated type-1 cytokine IFNγ and type-2 cytokine after IL-18 stimulation. The findings described here support the dual role of IL-18 on Th1 and Th2 cytokine production involving DC.

Human myelomonocytic KG-1 cells were grown in culture medium (IMDM, 10% FBS, 1% Glutamine, and 1% penicillin/streptomycin). Cell cultures were passaged when they reached a density of 2×10⁶ cells/ml and diluted to density of 4×10⁵ cells/ml.

PBMC were separated from heparinized buffy coat (interstate blood bank, TC) by standard gradient centrifugation with Ficoll-Hypaque (Amersham Pharmacia, Uppsala, Sweden). PBMCs were harvested and washed twice and were incubated with anti-human CD 14⁺ Mabs conjugated microbeads (Miltenyi Biotec GmbH, CA) for 15 min on ice, washed twice and passed over a column in strong magnetic field using the VARIO MACS technique as recommended by the manufacturer (Miltenyi Biotec GmbH, CA). Purity of monocytes was determined by flow cytometry. The cells in the preparation were found to be >95% CD14⁺.

To induce DC differentiation, the CD14⁺ monocytes (5×10⁵ cells/ml) were cultured in complete medium (RPMI/10% FBS), 1,000 IU/ml GM-CSF (Biosource International, CA), and 1,000 IU/ml IL-4 (Biosource Intl, CA), at 37° C. in 5% CO₂ for 6 days. The culture medium and cytokines were renewed every other day. On day 6 cells were harvested and transferred to fresh 12-well plates in complete culture medium with 10 ng/ml hTNFα, 200 ng/ml hIL-18 for another 4 days. Cells surface marker was analyzed using flow cytometric methods. Statistical comparisons between control and cytokine treated groups were performed using student's t-test. Values of p<0.05 were considered statistically significant.

### Methods of Preparing IL-18 Matured DC

The invention provides for the use of an effective amount of IL-18 to increase or mobilize mature dendritic cells in vivo, for example, in the patient's peripheral blood or other tissue or organs, such as the spleen. By increasing the quantity of the patient's mature dendritic cells, such cells may themselves be used to present antigen to T cells. For example, the antigen may be one that already exists within the patient, such as a tumor antigen, or a bacterial or viral antigen. IL-18 may be used, therefore, to boost the patient's lymphocyte-mediated (e.g., T cell and B cell mediated) or myeloid-mediated immune response to the already present antigens thus potentially enabling a more effective antigen-presentation to the patient's T cells. Alternatively, IL-18 may be administered prior to, concurrently with or subsequent to administration of an antigen to a patient for immunization purposes. Thus, as a vaccine adjuvant, IL-18 can generate large quantities of dendritic cells in vivo to more effectively present the antigen. The overall response is a stronger and improved immune response and more effective immunization to the antigen.

The therapeutic dendritic cell compositions of the invention are prepared using either a precursor dendritic cell or an immature dendritic cell. In these embodiments, the composition is incubated ex vivo under conditions that allow for maturation of the immature dendritic cell prior to administering the composition to the patient.

A precursor dendritic cell or an immature dendritic cell may be obtained using methods known in the art. U.S. Ser. No. 09/853,300 (published as U.S. 20020048583) discloses methods of isolating dendritic cells, the teachings of which are incorporated herein by reference.

For any of the ex vivo methods of the invention, peripheral blood progenitor cells (PBPC) and peripheral blood stem cells (PBSC) are collected using apheresis procedures known in the art such as by ficoll-hypaque (Histopaque 1.077, commercially available from Sigma, St. Louis, Mo.) gradient centrifugation, and viably frozen using an automated cell freezer (commercially available from Gordinier Electronics, Roseville, Mich.) in RPMI (commercially available from Life Technologies, Frederick, Md.) containing 40% human protein serum (commercially available from Gemini Bio-Products, Woodland, Calif.) and 10% DMSO (Sigma). The cells are stored in the vapor phase of liquid nitrogen until used. DNA can be prepared from a portion of the cells and used for molecular HLA typing. See also, for example, Bishop et al., Blood, vol. 83, No. 2, pp. 610-616 (1994). Briefly, PBPC and PBSC are collected using conventional devices, for example, a Haemonetics Model V50 apheresis device (Haemonetics, Braintree, Mass.). Four-hour collections are performed typically no more than five times weekly until, for example, approximately 6.5×10⁸ mononuclear cells (MNC)/kg patient are collected. The cells are suspended in standard media and then centrifuged to remove red blood cells and neutrophils. Cells located at the interface between the two phases (also known in the art as the buffy coat) are withdrawn and resuspended in HBSS. The suspended cells are predominantly mononuclear and a substantial portion of the cell mixture are early stem cells.

One method of selecting dendritic cells (DC) is by a process of negative selection. To do this, DC precursors are prepared from freshly-thawed PBMC by negative selection using immunomagnetic bead depletion. Specifically, PBMC were placed into a tube and incubated on ice for 30 min. with mouse anti-human CD3, CD 16, and CD19 antigens (commercially available from Caltag, Burlingame, Calif.). Excess antibody is removed by washing the cells with phosphate buffered saline containing 1% of bovine serum albumin (PBS/0.1% BSA), and the washed cells are next incubated with Pan Mouse IgG immunomagnetic beads (commercially available from Dynal, Lake Success, N.Y.) for 30 min. on ice. The tube containing the cells plus specific mouse anti-human antigens and the Pan Mouse IgG immunomagnetic beads is placed against a magnet to remove the cell:bead complexes. The cells that bound to the magnet are either T cells, B cells, or Natural Killer (NK) cells. Accordingly, the supernatant contained the lineage-depleted DC precursors (i.e., the monocytes remaining in the fluid in the tube not expressing CD3, CD 16, or CD19 antigens and so not bound by the magnet). These negatively selected cells are typically approximately 70% pure monocytes as characterized by Flow cytometry using a broad CD marker panel (see Table I above) were collected.

Alternatively, DC precursors are collected by positively selecting either CD 14+ cells from PBMC or CD34+ precursor cells from PBMC, bone marrow, cord blood or other suitable source using, e.g., monoclonal antibodies against CD14 or CD34 conjugated to magnetic beads (e.g., available from Miltenyi Biotech, Auburn, Calif.).

Next, the selected cells are washed, resuspended in culture medium containing human serum (from a person with blood type AB), GM-CSF (1000 U/mil) and IL-4 (1000 U/mil) (both commercially available from R & D Systems, Minneapolis, Minn.) and cultured at 37° C. in 5% CO₂ at 0.5×10⁶cells/well in 24 well plates for four days.

For the growth and culture of dendritic cells, a variety of growth and culture media can be used, and the composition of such media can be readily determined by a person having ordinary skill in the art. Suitable growth media are solutions containing nutrients or metabolic additives, and include those that are serum-depleted or serum-based. Representative examples of growth media are RPMI, TC 199, Iscoves modified Dulbecco's medium (Iscove, et al., F.J. Exp. Med., 147:923 (1978)), DMEM, Fischer's, alpha medium, NCTC, F-10, Leibovitz's L-15, MEM and McCoy. Particular examples of nutrients that will be readily apparent to the skilled artisan include, serum albumin, transferrin, lipids, cholesterol, a reducing agent such as 2-mercaptoethanol or monothioglycerol, pyruvate, butyrate, and a glucocorticoid such as hydrocortisone 2-hemisuccinate.

The cultured cells are immature dendritic cells by day four. A typical chronological pattern of surface expression of numerous cell surface antigens analyzed by flow cytometry is shown in Table I below.

TABLE I

| Human Dendritic | Day 0 | Day 4 | Day 7 |
|---|---|---|---|
| Cell Surface Markers | Monocytes | immature DC | mature DC |
| Marker (all cells) | | | |
| HLA-DR | 70-85% | 80-85% | 95-99% |
| HLA-ABC | 70-85% | 85-90% | 95-99% |
| CD3 | 1-5% | ND | ND |
| CD4 | 2-3% | ND | ND |
| CD8 | 2-3% | ND | ND |
| CD16 | 3-15% | 15-40% | 0.5-5% |
| CD19 | 5-10% | ND | ND |
| CD14 | 75-80% | 0.4-0.5% | 0.1-0.2% |
| CD11c | 75-80% | 95-99% | 99-100% |
| Marker (gated on DC) | | | |
| CD86 | 85-90% | 40-70% | 95-99% |
| CD80 | 30-50% | 55-80% | 85-90% |
| CD40 | 40-50% | 55-60% | 55-60% |
| CD83 | 10-15% | 10-15% | 55-60% |
| CD32 | 89-98% | 70-95% | 40-45% |
| CD64 | 92-99% | 28-60% | 4-10% |

On about the fourth day of culture, the cells are pulsed with antigen (e.g., Prostate Specific Antigen (PSA)) and incubated for an additional period of time, typically three days. The selection of antigen for pulsing will depend on the intended use of the matured DC. For example, if the DC cells are to be used to generate a T cell response to PSA, the DC cells are pulsed with PSA antigen. If the DC are to used to fight infection, an antigen related to the pathogen will be used.

At a prescribed time point after antigen pulse, preferably eight hours after addition of the antigen, IL-18 is added to the culture in order to induce DC maturation. Other agents such as TNFα (10 μg/ml) and/or IFNα (50 μg/ml). The matured DC are typically harvested on the seventh day of culture, analyzed for phenotypic markers by flow cytometry.

DC precursors are collected by positively selecting either CD14+ cells from PBMC or CD34+ cells from PBMC, bone marrow or cord blood using monoclonal antibodies against human CD14 or CD34 conjugated to magnetic beads. Human Langerhans cells can be derived by exposing DC percursors to GM-CSF, IL-4 and/or TGF-\tilde{□}.

The process of inducing IL-18-matured DC is not limited to the exclusive use of human recombinant IL-18 (SEQ. ID. NO. 1) but includes biologic or chemical entities that induce interferon-gamma and bind and activate to IL-18R (IL-18R agonists). The collected CD80⁺/CD86⁺ cells are then exposed to an IL-18 compound alone or IL-18 compound in concurrent or sequential combination with one or more of the following cytokines: flt-3 ligand, GM-CSF, IFN-□, IFN-□, TNF-α, CD40 agonists, IL-3, IL-4, c-kit-ligand or GM-CSF/IL-3 fusion proteins. The precursor DC or iDC are allowed to differentiate and commit to cells of the dendritic lineage. The dendritic cells are collected and can either be (a) administered to a patient in order to augment the immune system and T-cell mediated or B-cell mediated immune responses to antigen, (b) exposed to an antigen prior to administration of the dendritic cells into a patient, (c) transfected with a gene encoding an antigen-specific polypeptide or (d) exposed to an antigen and then allowed to process and present the antigen, ex vivo, to T-cells collected from the patient followed by administration of the antigen-specific T-cells to the patient.

Other agents that induce DC maturation include toll like receptor ligands including unmethylated bacterial CpG DNA or synthetic oligonucleotides containing CpG motifs, double stranded viral RNA and members of the TNF superfamily of cytokines including, but not limited to CD40 and OX40 agonists.

### Disease Specific Antigens of the Invention

Some non-limiting examples of antigens associated with a disease include the prostate specific antigen (associated with prostate cancer), BRCA-1 and BRCA-2 antigens (associated with many adenocarinomas, including breast cancer, lung cancer, and pancreatic cancer), CA 125 (associated with ovarian cancer), aberrant myelin basic protein (associated with Alzheimer's disease), gp120 (associated with HIV infection and AIDS), MUC-1 (associated with breast cancer), EBNA-1 (associated with Epstein Barr Virus infection), CA 19.9 (associated with colorectal, stomach, and pancreatic cancers), and TAG-72 (associated with ovarian, stromal, and pancreatic cancers), p53 (associated with various cancers).

Thus, in certain preferred embodiments, the antigen is a tumor-associated antigen. A “tumor associated antigen” is an antigen in the patient's body that is made by tumor cells, and which may be presented on the tumor surface, or circulating, or both. Preferred tumor-associated antigens include, without limitation, CA125, PSA, MUC-1, CA19.9, and TAG-72. Generally from about 0.1 to about 50 μg antigen are used.

In certain preferred embodiments, the antigen is from a pathogen. A “pathogen” is an etiolytic agent capable of causing disease. Preferred pathogens include, without limitation, viruses (e.g. hepatitis B, hepatitis C, herpes, and HIV-1), viroids, bacteria, fungi, prions, and parasites.

GVHD

Because of its ability to generate dendritic cells, IL-18 also finds use in promoting the survival of transplanted tissue or organs. When allogeneic organs or other tissue is transplanted into a host the transfer includes stem cells, immature dendritic cells, and mature dendritic cells from the donor. These cells are called passenger cells and such cells can graft into the hematopoietic system of the host. Additionally, stem cells, immature dendritic cells, and mature dendritic cells from the host may graft to the donor organ or tissue. It is possible then to establish a tolerance between the graft and the host since the immature dendritic cells from the host and donor tissue interact with T-cells from the “other side.” Such interaction may include the deletion of T-cells that recognize the major histocompatability complex (MHC) that the dendritic cells express. In this way, the donor cells are “screened” so that they fail to recognize and react against the host (i.e., no graft versus host disease) and the host T-cells are screened so that they fail to recognize and react against the graft (i.e., no graft rejection). Thus, a mutual tolerance can be achieved, and the graft acceptance is improved. Administration of IL-18 or IL-18 matured DC to the host or donor prior to transplantation would provide for increased numbers of dendritic cells in such host or donor and permit increased tolerance and survival of the graft.

Disease Caused by Pathogens

A vaccine consisting of IL-18-treated DC loaded with antigen from the pathogen (optionally further comprising or including administration of a DNA vaccine), or IL-18 in combination with other cytokines, including but not limited to those disclosed herein.

### Methods of Administering IL-18 Matured DC

By “autologous” is meant having identically matched MHC loci (both class I and class II). Thus, an identical sibling can provide autologous dendritic cells for a patient. Similarly, a close relative can provide autologous dendritic cells for a patient, so long as the patient and the close relative have identically matched MHC loci.

In certain preferred embodiments, if the patient to whom the composition of the invention is administered already had an immune response to the antigen, following administration, the immune response is shifted predominantly from a helper to a cytotoxic T cell response, thus providing the patient, following administration, a therapeutic benefit.

Thus, in one non-limiting example, a patient of the invention with prostate cancer may already have either antibodies that are specific for prostate specific antigen (PSA) and/or helper T cells that are specific for PSA. However, following administration of the composition of the invention, the PSA of the composition is internalized and presented on antigen presenting cells in such a way (e.g., in context of MHC class I) that cytotoxic T cells that are specific for PSA are stimulated, thereby providing the patient a therapeutic benefit as compared to the patient's condition prior to administration of the composition.

The systemic administration of IL-18 or IL-18 matured DC not only is effective as a vaccine adjuvant, but as discussed supra., is effective in augmenting an immune response against previously existing antigens.

An indirect effect of IL-18 on augmenting an immune response through cross-presentation of IL-18 treated, apoptotic or necrotic DCs by neighboring DCs is also included in the present invention.

The preparation and/or administration of matured DC preparations of the present invention can be accomplished by methods described herein or as known in the art, e.g., using sterile techniques and standard infusion techniques. Additionally, specialized devices for processing, preparing, and re-infusion of the cell preparations to patients are known in the art. Such ex-vivo or ex-corporeal methods for separating, treating, and recombining a patients blood cells with other blood components are well known in the art, e.g., as disclosed in WO 99/38380 and/or WO 00/62818, which are entirely incorporated by reference.

The breadth and scope of the invention will be further understood by examples disclosed hereinbelow.

### EXAMPLE I

### IL-18 Upregulates IL-1R-Related Protein and Other Receptor Expression

The functional IL-18R is composed of the binding chain α, IL-1Rrp (IL-1 receptor related protein), and non-binding chain β, AcPL (Accessory Protein-Like). Cells known to express IL-18R include activated T, B cells and NK cells (Nakamura, S., et al. Leukemia 14:1052, 2000). The fact of IL-18 increases IFNγ production by KG-1 cells and IL-18+IL12 synergistically induce IFNγ production by mouse dendritic cells suggest that IL-18 has activity on myeloid cells presumably through the IL-18R (Stober, D., et al. J. Immunol. 167:957, 2001).

KG-1 cells were incubated with GM-CSF/IL-4, IL12, IL-18 and TNFα for 6 days and subsequently stained by PE-conjugated IL-1Rrp Mab or isotype control antibody and data was collected by flow cytometry. Mabs recognizing the following antigens were used: CD83, CD86, CD80, DR, CD40, ICAM-1, mouse IgG1κ-PE, and mouse IgG1κ-FITC from BD PharMingen. IL-1Rrp antibody was from R & D System (Minneapolis, Minn.). KG-1 cells 5×10⁵ cells were stimulated with GM-CSF (1,000 IU/ml), IL-4 (1000 U/ml), recombinant human IL-12 (2 ng/ml, R & D, 309-1L, lot JB038111), recombinant human IL-18 (200 ng/ml, RDI, L089, lot 06993), and recombinant human TNFα (10 ng/ml, R&D). The culture medium and cytokines were renewed every other day. On day 6 and day 9, cells staining was performed on 1×10⁵ cells per sample and labeled at 4° C. for 30 min with FITC or PE labeled antibody. Flow cytometry was conducted on a FACSCalibur™ and analyzed using CELL Quest™ software (Becton Dickinson).

We found that the expression of IL-18Rrp is significantly upregulated by IL-18. To confirm that subsequent protein expression was similarly effected, we stimulated KG-I cells and analyzed IL-18Rrp protein expression by using flow cytometry.

Several other cell surface markers of mature dendritic cell (DC) related genes were up regulated by IL-18. These included, but were not limited to, CD83, LT, IL-13Ra, IL-8, ICAM-1, PGE2, NFkB, STAT4, CD69, IL-1ra, IL-2R, IL-4R, LPSbp, MCP-1, c3, TNFR2 and CSF-1R.

### EXAMPLE II

### Gene Expression Microarray of KG-1 Cells Stimulated by IL-18.

In order to better understand the role of IL-18 on myeloid compartment gene expression, a microarray analysis using IL-18 stimulated KG-1 cells derived samples was conducted.

KG-1 cells (1×10⁶/ml) were incubated with recombinant human IL-18 for 2, 4, and 8 hr at 200 ng/ml concentration. Total RNA was isolated with the Ultraspec RNA isolation system (BIOTECX) from cultured cells. The labeled probes were hybridized to a suitable blood type DNA chip (available, e.g., from Affymax). Hybridization of each sample was performed on two identical chips. Intensity of each clone was determined as the average intensity of the four corresponding spots and the coefficient of variation (CV) associated with each intensity value was calculated. If CV was >=50.0%, the corresponding intensity value was discarded. The average intensity of 15 plant genes was used as background. In this analysis, value of 29 was determined to be the background threshold and was applied to intensities <29 (if intensity <29, it was adjusted to 29). The pair-wise correlation of gene expression profile of all 8 samples was examined by scatter plot and the correlation coefficient was calculated. All 8 samples correlated with each other very well with correlation coefficient >=0.96.

For each clone, ratio of gene expression was calculated as the intensity of IL-18 treated sample divided by the intensity of control sample at each time point. The criteria to select genes with significant gene expression change with respect to IL-18 treatment was at least 2 fold. 35 out of 3958 clones passed the filtering and 8 genes were further selected from the 35 genes. The change in expression of 8 selected genes in IL-18 treated KG-1 cells was meastured at 2 hr, 4 hr and 8 hr time points. Ratios of the gene expression levels of treated samples to respective control samples were derived from normalized signal intensities on DNA microarray chips.

Enhanced display of IL-1 Rrp shown by flow cytometric analysis was confirmed by the microarray studies demonstrating that IL-18 upregulated IL-1Rrp gene and by RT-PCR (data not show). The microarray results showed that IL-18 up regulated cytokine and chemokine genes such as IL-8, LT, GRO-γ, cytokine receptors such as IL-18Rrp and IL-13Rα, cell activation marker CD69 and also upregulated signaling proteins gene such as c-myb, c-abl, TNFα inducible and IFN-gamma inducible protein gene expression (data not shown). Most unexpectedly, IL-18 caused the upregulation of CD83 gene expression, a well-defined marker for mature DCs.

### EXAMPLE III

### IL-18 Upregulates Expression of Dendritic Cell Maturation and Costimulatory Surface Molecules on KG-1 Cells

In order to confirm that IL-18 can induce the expression of DC maturation genes at the protein level, we stimulated KG-1 cells with GM-CSF+IL-4, IL-18, IL-12, and TNFα and conducted flow cytometric analyses for protein expression of various surface markers. On day 3, the expression of CD83 a hallmark phenotype for maturation of DCs was upregulated (MFI of control vs. IL-18 treated; 11±5.1: 19±4.5, n=3), IL-18 also up-regulated DR (521±149: 945±85) and ICAM-1 expression (61.4±15:116±28). On day 6, the expression of CD83, ICAM-1, and CD80 expression were up-regulated (data not shown).

The effect on KG-1 cells of incubation with GM-CSF/IL-4, IL-18, IL-12, TNFα and various cell surface makers at day 9 was measured. The cells were stained with FITC or PE-conjugated CD83, CD40, CD80, and CD86 and isotype control on day 9. The □ MFI are shown (control versus treated (solid versus profile)). Three independent experiments were performed and a representative one is shown.

IL-18 and TNFalpha but not IL-12 or GM-CSF+IL-4 increase expression of the costimulatory molecules CD80, CD86, CD40 and CD83 on day 9.

### IL-18 Upregulates Expression of Dendritic Cell Maturation and Costimulatory Surface Molecules on Human DCs

To confirm that upregulation of markers occurs in primer cells, we stimulated human CD 14⁺ monocytes with GM.CSF/IL-4 for 6 days and then stimulate cells with IL-18 for another 4 days. The expression of CD83, CD80 and ICAM-1 were assayed using labeled antibodies by flow cytometry as described elsewhere.

IL-18 treatment increased maturation related markers CD83 as compared to the unstimulated control cells for: CD83, from 35±2 to 41±1.5; ICAM-1, from 140±3 to 172±3; and CD80, from 137±2 to 172±3 (units are arbritary).

### CD83, CD40 and CD80 Expression Induced by IL-18 is Independent of Endogenous TNFalpha

TNFalpha is well known DC maturation factor (Feuerstein, B., et al. J Immunol Methods 245: 15-29, 2000; Lapointe, R., et al. Eur J Immunol 30: 3291,2001). TNFalpha induces expression of DC maturation markers and costimulatory molecules. In order to establish that IL-18 induced CD83, CD40, CD86 and CD80 protein expression was not due to endogenous TNFα, we stimulated KG-1 cells in the presence of anti-TNFα neutralization antibody cA2 (infliximab, Centocor, Inc, Malvern, Pa.).

KG-1 cells were incubated with IL-18 or TNFα and with/without anti-TNFα antibody cA2. The cells were subsequently stained with FITC or PE-conjugated CD83, CD40, CD80, and isotype control on day 9. The cell surface protein expression was detected by flow cytometry as described above. The CD83 expression induced by exogenousTNFα was blocked whereas CD83 expression induced by IL-18 was only partially inhibited by anti-TNFα antibody. The results indicate that the expression of cd40 and cd80 by il-18 is independent on endogenous tnfα but can be stimulated by exogenously suppled TNFalpha. The MFI are shown. Two independent experiments were performed.

### EXAMPLE IV

### IL-18 Induction of Cytokine Expression

IL-18, first described as an IFNgamma inducing factor, can also induce the type 2 cytokines IL-4 and IL-13 by T cells, NK cells, mast cells and. Human alveolar macrophages and DC subpopulations may also have the capacity to produce IL-13 in certain circumstances but the direct effect of IL-18 on IL-13 production by human monocytic cells has not been studied. In human systems, lymphoid DCs were shown to generate type 2 response, while myeloid DCs to generate a type 1 response (Nakanishi, K., et al. Ann Rev. Immunol. 19: 423, 2001.

KG-1 cells were incubated with cytokine at the indicated concentrations. The supernatant was collected on day two and the level of cytokine was tested using a capture-sandwich immunoassay developed for the Luminex instrument (Luminex, Austin, Tex.). The assay used LabMAP™ cytokine capture microspheres including anti-human IL-12P40, IL-12p70, IL-13, IL-15, IFNγ and LabMAP biotin-conjugated cytokine detection antibodies (Luminex, Austin, Tex.). Streptavidin-R-Phycoerythrin.

KG-1 cells are CD8α negative (date not shown). IL-18 increases IFNγ by about 5-fold and IL-13 production from undetectable levels to over 400 pg/ml in KG-1 cells.

TNFalpha did not increase IFNgamma levels produced by KG-1 cells nor did the combination of GM-CSF and IL-4, or IL12 alone. IL-13 production was increased by TNFalpha but not GM-CSF/IL-4 combination. Il-13 production was also stimulated by IL-12 alone but not to the level produced by IL-18. RT-PCR results showed that IL-18 caused a direct increase in IL-13 gene expression on KG-1 cells after 3 hour stimulation (data not shown) and that IL-18 treatment had no effect on IL-12 p40 or p70, nor IL-15 production (data not shown).

### EXAMPLE V

### IL-18 Inhibits Endocytosis

Dendritic cells express a number of receptors that mediate endocytosis. These include Fc receptor, the Mac-1 molecular. Immature DCs have a high endocytotic activity. Endocytosis by monocytes and immature myeloid DCs is predominantly mediated by the macrophage mannose receptor (MMR) and can be measured using FITC-dextran uptake followed by flow cytometry (Kato, M., et al. Int Immunol 12:1511, 2000).

KG-1 cells were treated with GM-CSF/IL-4, IL-12 alone, IL-18 alone or TNFalpha. KG-1 cells, after 6 days stimulation with cytokines, were suspended in medium and incubated with 1 mg/ml of FITC-dextran (Mr+40,000; Sigma) for 30 min at 4 and 37° C. Cells then were washed 3 times with ice-cold PBS, 0.1% BSA and 0.01% NaN₃. The uptake was calculated as the change in MFI between cell samples incubated at 37 and in FITC-dextran for 30 min at 4 or 37° C. FITC-dextran uptake by the cytokine pre-treated KG-1 cells was compared. Cells were washed 3 times and uptake uptake was measured by flow cytometry. Results were calculated as the change in MFI between cell samples incubated at 37° C. and 4° C. The results are representative of two similar experiments.

Overall level levels of endocytosis are downmodulated as DCs mature. Thus, the FITC-dextran endocytosis assay, is further evidence of DC differentiation. Aftere a six day stimulation of KG-1 cells with cytokines, both IL-18 and TNFα inhibited FITC-dextran uptake by KG-1 cells while IL-12 and GM-CSF+IL-4 had no effect.

### EXAMPLE VI

### IL-18 Increases Mixed Lymphocyte Reaction

Maturate DCs express high levels of costimulatory and adhesion molecules that favour T cell stimulation and T cells proliferation. IL-18 stimulated DCs ability to induce human lymphocyte proliferation is another measure of a more mature DC phenotype.

DCs induced allogeneic MLR: CD14⁺ monocytes were treated with GM-CSF (1,000 IU/ml), IL-4 (1,000 IU/ml) for 6 days and were stimulated with IL-18 (200 ng/ml) or TNFα (10 ng/ml) for another 4 days. Cells were treated with mitomycin C (25 μg/ml) in 37° C. for 30 min and were washed three times with PBS as stimulator cells.

As stimulator cells, DCs (1×10⁴) were plated in triplicate in 96-well plates (Costar, Cambridge, Mass.) and co-cultured with 1×10⁵/well of autologous or allogeneic lymphocytes used as responder cells. IL-18 treated DCs were cocultured with CD14 depleted human PBMCs at ratio of 1:10. Cultures were maintained at 37° C. with 5% CO₂ for 4 days and cell proliferation was measured by ATPLite-M assay (Packard BioSience B. V. Netherlands).

IL-18 treated DCs were strong stimulators of allogeneic T proliferation. The cell proliferation was measured using ATP-Lite assay. Data is representative of two similar experiments. CD14⁺ human monocytes were cultured GM-CSF/IL4 for 6 days and then stimulated with IL-18 and TNFα for another five days. The cells were treated with mitomycin-C for 30 min and co-cultured with allogeneic or autologous T cells (DC: T, 1:10) for four days. KG-1 cells treated with IL-18 and TNFα enhancement of lymphocytes proliferation was statistically significant.

Table of Abbreviations
Abs antibodies, polyclonal or monoclonal
CC cys-cys type chemokine
CXC cys-Xcys type chemokine
DC dendritic cells
FDC follicular dendritic cell
GM-CSF granulocyte-macrophage colony stimulating factor
ICAM intercellular adhesion molecule
IDC interdigitating dendritic cell
iIDC immature interdigitating dendritic cell
IFN interferon
Ig immunoglobulin
IgA immunoglobulin A
IgG immunoglobulin G
IL interleukin
IL-18R interleukin-18 receptor
IL-18Rrp interleukin-18 receptor related protein
Mab monoclonal antibody
MFI mean fluorescence intensity
MRL Mixed Lymphocyte Reaction
NK natural killer cells
PBMC peripheral blood mononuclear cells
PBPC peripheral blood progenitor cells
PBSC peripheral blood stem cells
Tb 1 T helper cell type
Th 2 T helper cell type 2
TNF tumor necrosis factor

1. A method for augmenting an immune response in a patient, comprising
(a) administering an amount of at least one composition comprising molecules having at least one IL-18 biological activity to the patient sufficient to generate an increase in the number of the patient's dendritic cells.

2. A method according to claim 1, further comprising
(b) administering at least one composition comprising at least one selected from the group consisting of flt3-ligand, GM-CSF, IL-4, TNF-α, IL-3, c-kit ligand, and fusions of GM-CSF and IL-3.

3. A method for augmenting an immune response in a patient having an infectious disease, comprising
(a) administering IL-18 to said patient in an amount sufficient to generate an increase in the number of the patient's dendritic cells.

4. A method according to claim 3, further comprising
(b) administering one or more of the molecules selected from the group consisting of flt3-ligand, GM-CSF, IL-4, TNF-α, IL-3, c-kit ligand, and fusions of GM-CSF and IL-3.

5. A method according to claim 3, wherein the infectious disease is HIV.

5. A method for augmenting an immune response in a patient having a cancerous or neoplastic disease, comprising
(a) administering IL-18 in an amount sufficient to generate an increase in the number of the patient's dendritic cells.

7. A method according to claim 6, further comprising the step of administering one or more of the molecules selected from the group consisting of flt3-ligand, GM-CSF, IL-4, TNF-α, IL-3, c-kit ligand, and fusions of GM-CSF and IL-3.

8. A preparation of dendritic cells having at least two cell surface markers selected from the group consisting of CD1a, HLA-DR and CD86, produced by contacting hematopoietic stem or progenitor cells with IL-18.

9. A dendritic cell preparation according to claim 8 produced further by contacting the hematopoietic stem or progenitor cells with a molecule selected from the group consisting of flt3-ligand, GM-CSF, IL-4, TNF-α, IL-3, c-kit ligand, and fusions of GM-CSF and IL-3.

10. An antigen-expressing dendritic cell population produced by the process of (a) contacting hematopoietic stem or progenitor cells with IL-18 in an amount sufficient to generate a dendritic cell population; (b) either (i) exposing the dendritic cells to an antigen-specific peptide or (ii) transfecting the dendritic cells with a gene encoding an antigen-specific peptide; (c) allowing the dendritic cells to process and express the antigen; and (d) purifying the antigen-expressing dendritic cells.

11. A dendritic cell population according to claim 10 wherein step (a) of the process further comprises contacting the hematopoietic stem or progenitor cells with a molecule selected from the group consisting of GM-CSF, IL-4, TNF-α, IL-3, c-kit ligand, and fusions of GM-CSF and IL-3.

12. A method of driving hematopoietic stem or progenitor cells to a dendritic cell lineage comprising contacting such hematopoietic stem or progenitor cells with flt3-ligand.

13. A method of preparing an antigen-presenting dendritic cell population comprising the steps of: (a) contacting hematopoietic stem or progenitor cells with IL-18 in an amount sufficient to generate a dendritic cell population; (b) either (i) exposing the dendritic cells to an antigen-specific peptide or (ii) transfecting the dendritic cells with a gene encoding an antigen-specific peptide; (c) allowing the dendritic cells to process and express the antigen; and (d) purifying the antigen-expressing dendritic cells.

14. A method according to claim 13, wherein step (a) further comprises contacting the hematopoietic stem or progenitor cells with a molecule selected from the group consisting of flt3-ligand, GM-CSF, IL-4, TNF-α, IL-3, c-kit ligand, and fusions of GM-CSF and IL-3.

15. A method of preparing antigen-specific T cells comprising the steps of: (a) contacting hematopoietic stem or progenitor cells with IL-18 in an amount sufficient to generate a dendritic cell population; (b) either (i) exposing the dendritic cells to an antigen-specific peptide or (ii) transfecting the dendritic cells with a gene encoding an antigen-specific peptide; (c) allowing the dendritic cells to process and express the antigen; and (d) allowing the dendritic cells to present the antigen to T cells.

16. A process of preparing a matured dendritic cell comprising:
obtaining a sample of biological fluid containing stem-cells from a host;
separating a stem cell-containing biological fluid sample into a substantially stem cell-depleted portion, and stem cell-enriched portion;
culturing said stem cell-enriched portion with IL-18 to promote dendritic cell maturation; and
reinfusing said matured dendritic cells into said host.

17. A process of claim 16 wherein the separating step comprises:
providing an additive and magnetic particles, wherein the additive binds to the stem cell and the magnetic particles bind the additive; and
separating the stem cell-containing biological fluid sample by applying a first and a second force, wherein the first force is a magnetic force and the second force is a mechanical force.

18. A composition containing IL-18 matured DC.

19. A method of administering a composition containing IL-18 matured DC comprising:
infusing a preparation of cells into a patient intravenously.
